(19) United States
(12) Plant Patent Application Publication
(10) Pub. No.: US 2004/0128722 P1
(43) Pub. Date: Jul. 1, 2004

(54) Torenia plant named 'Sunrenilahopas'

(76) Inventor: Kazunari Iwaki, Ohmihachiman-shi (JP)

Correspondence Address: BURNS, DOANE, SWECKER & MATHIS, L.L.P., P.O. Box 1404, Alexandria, VA, 22313-1404, US

(21) Appl. No.: 10/327,944

(22) Filed: Dec. 26, 2002

(51) Int. Cl. A01H 5/00

(52) U.S. Cl. PLT/263

(57) ABSTRACT

Disclosed herein is a torenia plant having large flowers, yellowish white petals with yellow blotches and moderate red purple floral tube. The plant has a semi-decumbent growth habit, medium branching and a great profusion of blooms. The whole bush remains in bloom for a considerable period of time. The plant grows and has flowers ordinarily in the shade of trees.

### BACKGROUND OF THE VARIETY

The present invention relates to a new and distinct variety of torenia plant obtained from artificial doubling of ‘SUNRENIHOPAS’ (Torenia hybrid).

The torenia is a very popular plant and is used for flower bedding and potting in the summer season. There are only a few varieties of the torenia plant which have a semi-erect growth habit, medium branching and a great profusion of blooms.

Accordingly, this invention is aimed at obtaining a new variety having large flowers, yellowish white flower petal with yellow eye color, moderate red purple floral tube, semi-erect growth habit, medium branching and a great profusion of blooms.

The new variety of torenia plant according to this invention originated from artificial doubling of ‘SUNRENIHOPAS’, and we previously filed in Japan [‘SUNRENIHOPAS’ The Seeds and Seedlings law Registration No. 9823] and have not applied in United State of America and so on. In September 1998, the cuttings of ‘Sunrenihopas’ were treated with 0.1% colchicines solution, which were propagated by cutting on the peat at the Omi R&D Center, Flower Division, Suntory Ltd., located at 863-1, Aza-Iketani, Omori-cho, Yokaichi-shi, Shiga-ken, Japan. After two months, some surviving plantlets were transplanted in pots. In December 1998, colchiploids were obtained from the cultivation. The discovered torenia plant was propagated by cutting and was then grown in a bed and pots in a trial. The botanical characteristics of the plant were examined using the parent variety ‘Sunrenihopas’ and the similar variety ‘Sunrenilapiho’ for comparison. As a result, it was concluded that this discovered torenia plant is distinguishable from any other variety, whose existence is known to us, and is uniform and stable in its characteristics. Then this new variety of torenia plant was named ‘Sunrenilahopas’ (Torenia hybrid).

In the following description, the color-coding is in accordance with the Horticultural Color Chart of The Royal Horticultural Society, London, England (R.H.S. Color Chart). A color chart based on The Japan Color Standard for Horticultural Plant (J.H.S. Color Chart) is also added for reference.

‘SUNRENIHOPAS’ used as the parent of the variety ‘SUNRENILAHOPAS’ has filed in Japan [‘SUNRENIHOPAS’ The Seeds and Seedlings law Registration No.9823] and have not applied in United State of America and so on. The botanical characteristics of the torenia plant ‘SUNRENIHOPAS’ as follows.

Plant:

Growth habit.—Semi-erect.

Plant height.—15 cm.

Stem:

Diameter.—2.2 mm.

Anthocyanin pigmentation.—Present.

Branching.—Medium.

Pubescence.—Sparse.

Length of internode.—4.0 mm.

Leaf:

Shape of blade.—Lanceolate.

Length.—2.5 cm.

Width.—1.7 cm.

Depth of incision.—Medium.

Pubescence of upper side.—Sparse.

Flower:

Diameter.—2.2 cm.

Height.—2.8 cm.

Color of floral tube.—Bright reddish purple (R.H.S. 78B, JHS 8905).

Color of petal.—Single color; white (R.H.S. 155D, JHS -01).

Yellow eye color.—Present.

Calyx.—1.5 cm in length.

Anthocyanin pigmentation of calyx.—Present.

Peduncle.—1.4 mm in thickness; and 1.9 cm in length.

Reproductive organs.—1 pistil and 4 stamens.

Flowering duration.—Medium.

Physiological and ecological characteristics: Medium resistance to diseases and pests, Medium tolerances to heat and cold. The plant grows and has flowers ordinarily in the shade of trees.

‘SUNRENILAPIHO’ used as the comparison variety for the new variety ‘SUNRENILAHOPAS’ have previously filed in Japan [‘SUNRENILAPIHO’ The Seeds and Seedlings law Registration No.9825] and United State of America [TORENIA PLANT ‘SUNRENILAPIHO’ (U.S. Pat. No. Plant 12,105)] and so on. ‘SUNRENILAPIHO’ has a semi-erect growth habit, and bi-colored white and vivid reddish-purple flower petals without yellow blotches. The new variety has medium branching, and forms great profusion of blooms with the entire plant remaining in bloom for a considerable period of time. The main botanical characteristics of ‘SUNRENILAPIHO’ are as follows.

Plant:

Growth habit.—Semi-erect.

Plant height.—15 cm.

Stem:

Diameter.—Approximately 2.7 mm.

Anthocyanin pigmentation.—Present, and dark (RHS 166A).

Branching.—Medium.

Pubescence.—Sparse.

Length of internode.—5.5 cm.

Color.—Moderate yellow green (RHS 143C, JHS 3709).

Leaf:

Phyllotaxis.—Opposite.

Shape of blade.—Lanceolate.

Length.—3.8 cm.

Width.—3.2 cm.

Margin.—Dentate.

Color. (upper side).—RHS 138A when immature and RHS 137A when mature.

Color. (reverse side).—RHS 138B when immature and RHS 137C when mature.

Pubescence of upper side.—Sparse.

Flower:

Flower form.—Globose.

Diameter.—Long (approximately 33 mm) across the flower face parallel to the horizontal plane.

Height.—Medium to Long (approximately 35 mm).

Color of floral tube.—vivid reddish-purple (RHS 80A, JHS 8906).

Color of upper bilabiate petals.—Pale purplish-white (RHS 76D) on the upper surface and on the lower surface.

Color of intermediate bilabiate petals.—RHS 80A in the center with white margins on the upper surface and RHS 82A with white margins on the lower surface.

Color of lowest bilabiate petals.—RHS 78B towards the base except for a white margin and white towards the tip on the upper surface and the lower surface.

Color pattern of bilabiate petal.—Bi-colored.

Yellow eye color.—Absent.

Calyx.—Medium. (approximately 19 mm) in length.

Anthocyanin pigmentation of calyx.—Present towards the tip.

Peduncle.—1 Medium (approximately 1.5 mm) in thickness; and Medium (22 mm) in length.

Reproductive organs.—1 pistil and 4 stamens.

Anther color.—White.

Flowering time.—Late, early summer to mid-autumn.

Physiological and ecological characteristics: Moderate resistance to heat, cold, diseases and pests. Severe frost may be harmful to the plant. The resistance to powdery mildew is moderate.

### SUMMARY OF THE VARIETY

The new variety of torenia plant named ‘SUNRENILAHOPAS’ has a semi-erect habit, large flowers, yellowish white flower petal with yellow eye color, moderate red purple floral tube, medium branching and a great profusion of blooms.

The new variety of torenia plant named ‘SUNRENILAHOPAS’ have large flowers, and thus is very different from a similar variety, SUNRENIHOPAS’. The new plant has yellowish white single color flower petal with yellow eye color, and thus is very different from a similar variety, SUNRENILAPIHO’ having pale purplish-white upper bilabiate petals, vivid reddish purple intermediate bilabiate petals and bright reddish purple lowest bilabiate petals. The new plant has moderate red purple floral tube, and thus is very different from a similar variety, SUNRENILAPIHO’ having a vivid reddish-purple color.

### BRIEF DESCRIPTION OF THE DRAWINGS

FIG. 1 is a photograph giving a partial view of the new variety of torenia plant planted in a flower pot;

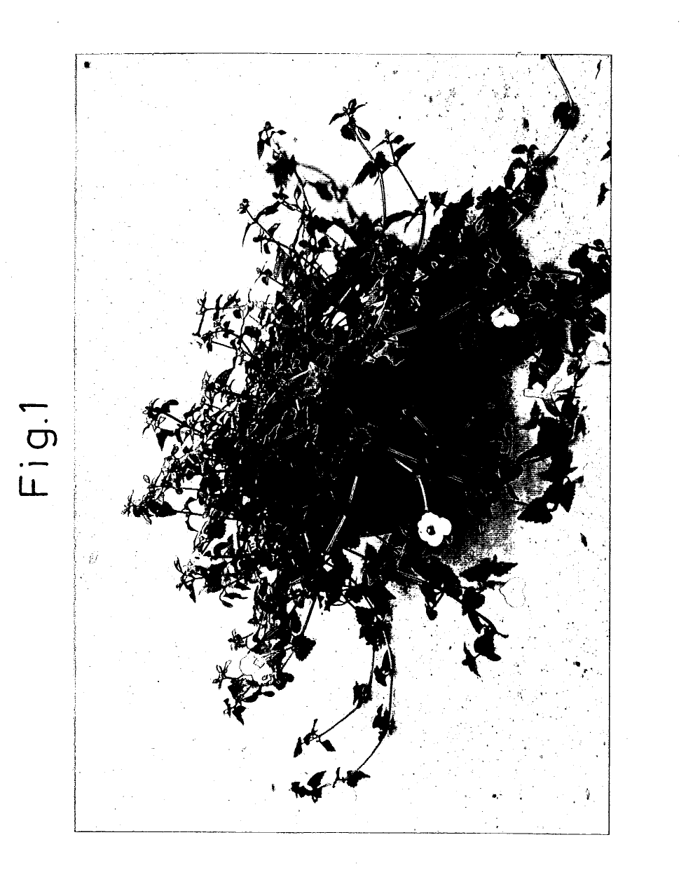

FIG. 2 is a photograph of flowers of the new variety of torenia plant ‘SUNRENILAHOPAS’ and those of the torenia plant ‘SUNRENILAPIHO’.

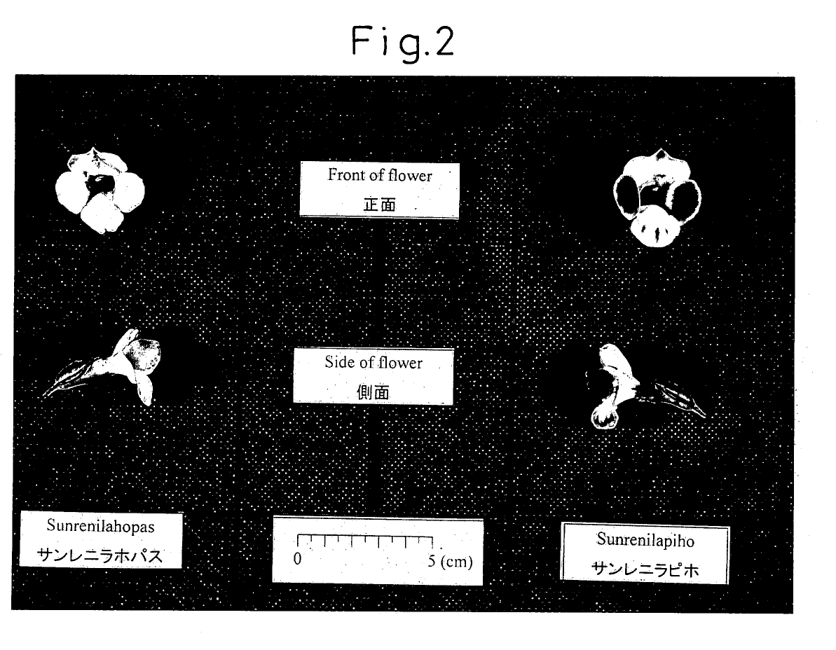

### DESCRIPTION OF THE VARIETY

The botanical characteristics of the new and distinct variety of torenia plant ‘SUNRENILAHOPAS’ are as follows. This investigation held in August at Yokaichi-shi, Shiga-ken, Japan. Plant materials, propagated by stem cuttings, examined in this investigation had been cultivated since May to November in 1999.

Plant:

Growth habit.—Semi-erect.

Plant height.—27 cm.

Stem:

Diameter.—2.0 mm.

Anthocyanin pigmentation.—Present. Light.

Branching.—Medium.

Pubescence.—Sparse.

Length of internode.—5.5 cm.

Leaf:

Shape of blade.—Oval.

Length.—2.5 cm.

Width.—2.5 cm.

Color. (upper side).—Dark green (R.H.S. 147A, JHS 3716).

Pubescence of upper side.—Sparse.

Thickness of petiole.—Medium.(1.5 mm).

Length of petiole.—Short.(0.5 cm).

Flower:

Inflorescence peduncles.—Axillary.

Flower form.—Globose.

Flower length.—Medium. (30 mm).

Flower width.—Medium. (30 mm).

Length of tubular.—Medium. (30 mm).

Color of floral tube.—Dull red purple. (R.H.S. 70A, JHS 9215).

Color of petal.—Single color; Yellowish white. (R.H.S. 156D, JHS 2701).

Yellow brotches of the petal.—Present.

Vertical line of the petal.—Present.

Wave of upper bilabiate petal.—Weak.

Calyx shape.—Deeply two lobed.

Calyx;Degree of development of wings.—Medium.

Calyx length.—Short to medium. (18 mm).

Anthocyanin pigmentation of calyx limb.—Absent.

Anthocyanin pigmentation of anther.—Absent.

Anther spur.—Present.

Peduncle thiskness.—Medium. (1.5 mm).

Peduncle length.—Short. (1.5 mm).

Spike existence.—Absent.

Spike; number of flower.—Few.

Reproductive organs.—1 pistil and 4 stamens.

Flowering duration.—Medium.

Physiological and ecological characteristics: Medium resistance to diseases and pests, high tolerances to heat and low tolerances to cold.

This new variety ‘SUNRENILAHOPAS’ of torenia plant is most suitable for flower bedding and potting, particularly in hanging pots or planters.

This new variety ‘SUNRENILAHOPAS’ was asexually reproduced by cutting.

The mother stocks of this variety are maintained as potted plants in a greenhouse, and as tissue culture materials in a laboratory

The plant of this new variety ‘SUNRENILAHOPAS’ is presently planted and maintained in the Omi R&D Center of SUNTORY FLOWERS LIMITED., located at 863-1, Aza-Iketani, Omori-cho, Yokaichi-shi, Shiga-ken, Japan.

1. A new and distinct variety of torenia plant, substantially as herein illustrated and described, characterized particularly as to novelty by (A) having a semi-decumbent growth habit and medium branching, (B) having large flowers and a great profusion of blooms and the whole bush remaining in bloom for considerable period of time, (C) flower petals are yellowish white with yellow blotch, (D) having moderate resistance to drought, diseases and pests and strong resistance to heat, and (E) growing and having flowers ordinarily in the shade of tree.
